(19) United States
(12) Patent Application Publication
(10) Pub. No.: US 2026/0199089 A1
(43) Pub. Date: Jul. 16, 2026

(54) PASSIVE SEAL FOR INTRODUCERS

(71) Applicant: Edwards Lifesciences Corporation, Irvine, CA (US)

(72) Inventor: Kurt Kelly Reed, Costa Mesa, CA (US)

(21) Appl. No.: 19/562,061

(22) Filed: Mar. 10, 2026

Related U.S. Application Data
(63) Continuation of application No. PCT/US2024/046137, filed on Sep. 11, 2024.
(60) Provisional application No. 63/582,380, filed on Sep. 13, 2023.

(51) Int. Cl. A61F 2/24

(52) U.S. Cl. CPC A61F 2/2436

(57) ABSTRACT

This disclosure is directed to introducers for delivering prosthetic devices to a desired implantation site, and to seal assemblies for such introducers. In some examples, the seal assemblies can include a passive seal movable between an open state and a closed state by the introduction and withdrawal of a guidewire that passes across the seal. In some examples, the seal assemblies can also include an internal guide channel that serves to align the guidewire with the seal. The seal assemblies disclosed herein are capable of maintaining an airtight seal both when the guidewire passes across the seal and when the guidewire is removed from the seal.

### CROSS REFERENCE TO RELATED APPLICATION

This application is a continuation of PCT Patent Application No. PCT/US2024/046137 filed on September 11, 2024, which claims the benefit of U.S. Provisional Application No. 63/582,380, filed September 13, 2023, each of these applications being hereby incorporated by reference herein in its entirety.

### FIELD

The present disclosure relates to introducer assemblies for implanting prosthetic devices and sealing assemblies for use with introducer assemblies.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

In some examples, a docking device can be implanted first within the native valve and can be configured to receive a prosthetic valve and secure (e.g., anchor) the prosthetic valve in a desired position within the native valve. For example, the docking device can form a more circular and/or stable anchoring site at the native valve annulus in which a prosthetic valve can be expanded and implanted. A transcatheter delivery apparatus can be used to deliver the docking device to the implantation site. In some examples, the transcatheter delivery apparatus can include an introducer that dilates the native vasculature of the patient to accommodate one or more components of the delivery apparatus, the prosthetic heart valve, and/or the docking device.

### SUMMARY

Described herein are delivery apparatus, and methods for implanting prosthetic heart valves. The disclosed delivery apparatus can, for example, include seal assemblies that provide a passive seal between one or more components of the delivery apparatus, such as an introducer and the outside environment. In some examples, the seal assemblies can further include an internal guide structure to facilitate the advancement of a guidewire across the seal. As such, the devices and methods disclosed herein can, among other things, overcome one or more of the deficiencies of typical delivery apparatus and methods for using typical delivery apparatus.

A delivery system can comprise a guide sheath and an introducer. In addition to these components, a delivery system can further comprise one or more of the components disclosed herein.

In some examples, the introducer can comprise a tubular shaft with an axially extending internal lumen.

In some examples, the introducer further comprises a nosecone at a first axial end of the tubular shaft and a handle at a second axial end of the tubular shaft.

In some examples the introducer assembly further comprises a seal assembly attached to an end portion of the handle.

In some examples, the seal assembly comprises a housing, a bore extending axially through the housing, and a sealing element disposed in the bore.

In some examples, the sealing element comprises a self-sealing channel, movable between an open state and a closed state.

In some examples, a delivery system can comprise a guide wire extending axially through the internal lumen of the introducer.

In some examples, the delivery system can further comprise a handle,

Certain examples concern an introducer assembly comprising a dilator. The dilator comprises a nosecone, a handle, a tubular shaft extending between the nosecone and a first axial end portion of the handle, and an internal channel extending axially through the nosecone, shaft, and handle. The introducer assembly also includes a seal assembly attached to a second axial end portion of the handle, the seal assembly comprising a housing, a bore extending axially through the housing, and a sealing element disposed in the bore. The dilator and the seal assembly are configured to receive a guidewire extending axially through the dilator and the seal assembly. The sealing element comprises a self-sealing channel configured to receive a guidewire, movable from a closed state to an open state when the guidewire is inserted into the self-sealing channel, and movable from the open state to the closed state when the guidewire is removed from the internal channel.

Certain examples concern a seal assembly for an introducer, comprising a seal housing. The seal assembly has an annular body comprising an internal circumferential groove and one or more radially tapered internal wall portions. The seal assembly also comprises an internal bore extending axially from a first end portion of the seal housing to a second end portion of the seal housing and a resilient sealing element disposed in the internal groove and comprising an axially extending slit circumferentially aligned with the internal bore. The axially extending slit is movable between an open state when a guidewire is inserted into the slit and a closed state when the guidewire is withdrawn from the slit. The seal assembly is configured to attach to an end portion of an introducer assembly comprising a hollow shaft with an internal channel.

Certain examples concern a delivery system comprising a guide catheter. The guide catheter comprises a tubular shaft having an internal lumen. The delivery system also includes a removable guidewire, an introducer, and a seal assembly. The guidewire is configured to extend through the internal lumen of the guide catheter. The introducer comprises a nosecone, a shaft, a handle, and an internal bore extending axially through the nosecone, the shaft, and the handle. The seal assembly comprises a seal housing, a seal element disposed within the seal housing, and one or more beveled guides defining a guide channel that extends axially through the housing of the seal assembly. The guide channel narrows from a first diameter at a first channel end to a second diameter smaller than the first diameter at a second channel end. The internal bore of the introducer and the guide channel of the seal assembly are configured to receive the guidewire. The seal assembly comprises an axially extending self-sealing channel that is configured to receive the guidewire. The self-sealing channel is movable from a closed state to an open state by introducing a wire into the self-sealing channel.

In some examples, a prosthetic heart valve comprises one or more of the components recited in Examples 1-22 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is a side elevation view of an introducer assembly according to one example.

FIG. 6 is a cross sectional schematic view of the introducer assembly of FIG. 5.

FIG. 7 is a side elevation view of a handle portion of a guide catheter according to one example, with the introducer assembly of FIG. 5 inserted into the guide catheter.

FIG. 8 is a side elevation view of the handle portion of the guide catheter of FIG. 7, with an introducer assembly and seal assembly according to one example.

FIG. 9A is a cross sectional schematic view of a seal assembly for an introducer according to one example.

FIG. 9B is a cross sectional schematic view of the seal assembly of FIG. 9A with a guidewire advanced across the seal.

FIG. 10A is a cross sectional schematic view of a seal assembly for an introducer according to another example.

FIG. 10B is a cross sectional schematic view of the seal assembly of FIG. 10A with a guidewire being advanced in a proximal direction.

FIG. 10C is a cross sectional schematic view of the seal assembly of FIG. 10A with a guidewire further advanced in a proximal direction.

FIG. 10D is a cross sectional schematic view of the seal assembly of FIG. 10A with a guidewire fully advanced across the seal.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

In some procedures, an introducer is advanced through a guide sheath to a desired implantation site. The guide sheath is generally sized to accommodate an implant being introduced, and the introducer is generally sized to closely match the diameter of the guide sheath. However, the introducer can also provide a pathway for the introduction of air embolisms into the guide sheath and/or the patient’s body. Therefore, care should be taken to seal the internal lumen during the delivery procedure, especially in examples where a dilator with a large internal lumen is used, including the removal of the guidewire and the introducer, to mitigate the possibility of introducing air into the system and/or a patient’s body.

Disclosed herein are methods to reduce or mitigate the possibility of introducing air embolism into the systema and/or a patient’s body, including several active sealing solutions and several passive sealing solutions.

Examples of prosthetic implants are disclosed in International Patent Application No. WO 2020/247907 and U.S. Patent No. 11,013,600, which are incorporated by reference herein.

One active sealing solution is to include a stopcock device at the proximal end of the introducer assembly, which allows the introducer assembly to be sealed after the guidewire is removed, such that the guide sheath can be aspirated before the introducer assembly is removed completely from the guide sheath. This allows for large diameter introducers to be used without risk of air embolism.

Another active sealing solution is to use a rotatable Touhy connector to form a gasket seal at the proximal end of the introducer assembly. This allows a seal to be formed or partially formed before the guidewire is removed, which allows for introducers to be used without risk of air embolism.

Passive seal assemblies can reduce or prevent the risk of air embolism without any additional actions required by the medical professional. Disclosed herein are various examples of valve introducers (sometimes called dilators) including a passive seal to mitigate risk of introducing air embolisms.

### The Disclosed Technology and Exemplary Examples

The introducers described herein are used for transseptal delivery of prosthetic devices, such as expandable stents, docking devices, and/or prosthetic heart valves. In the delivery of certain devices, including those disclosed in International Patent Application No. WO 2020/247907 and U.S. Patent No. 11,013,600, the devices disclosed herein can be used. Additionally or alternatively, the devices and methods disclosed herein can be used with various other implantable devices.

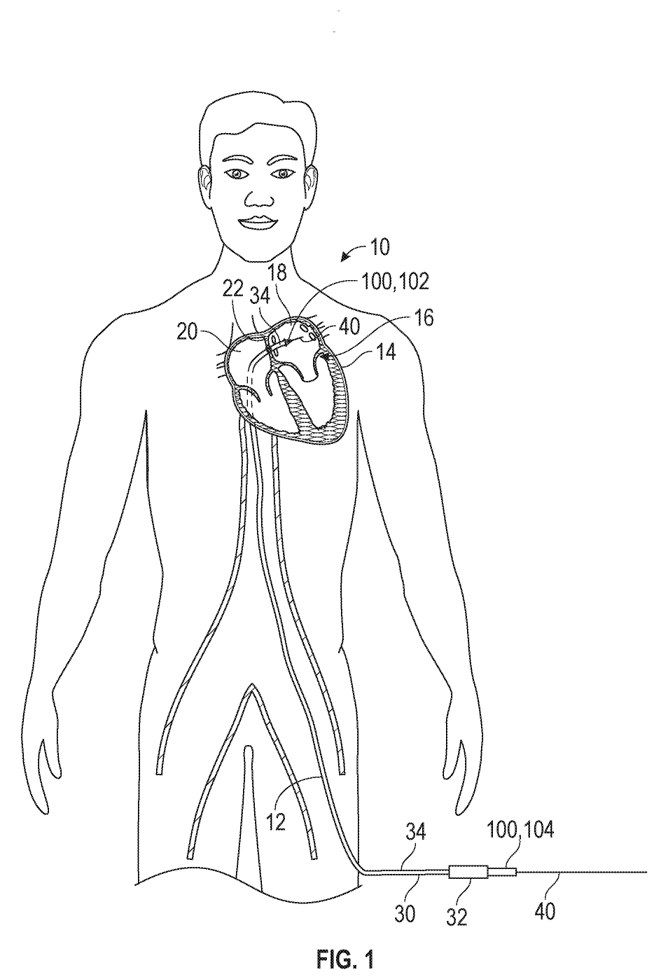

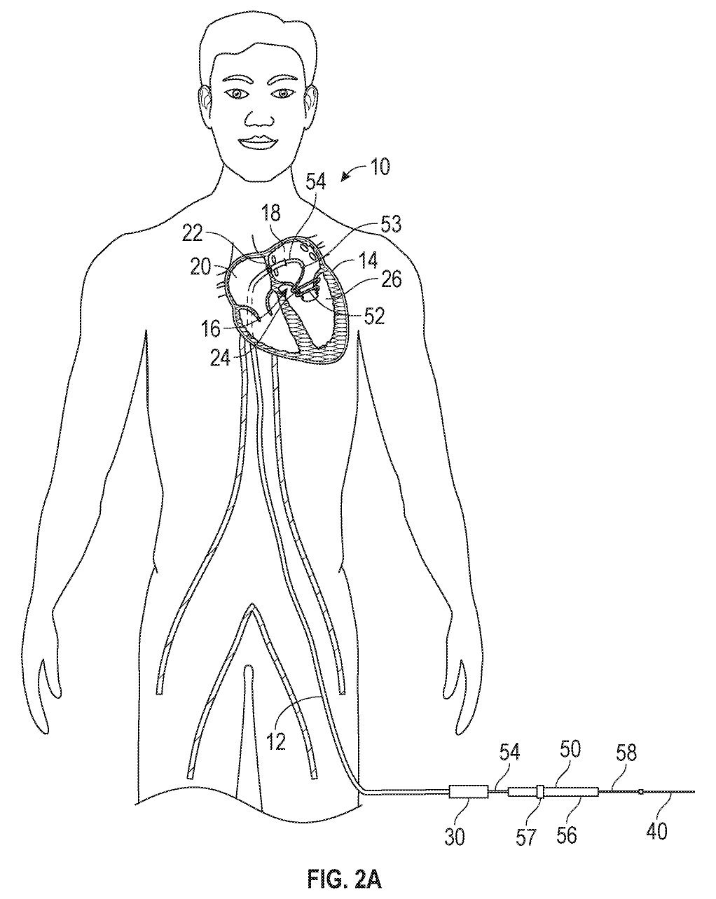

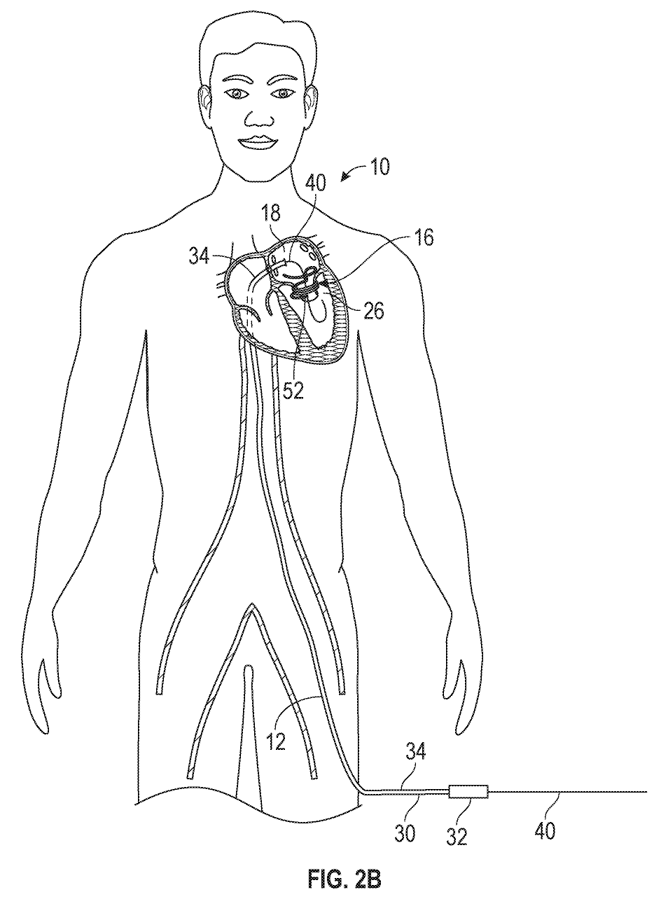

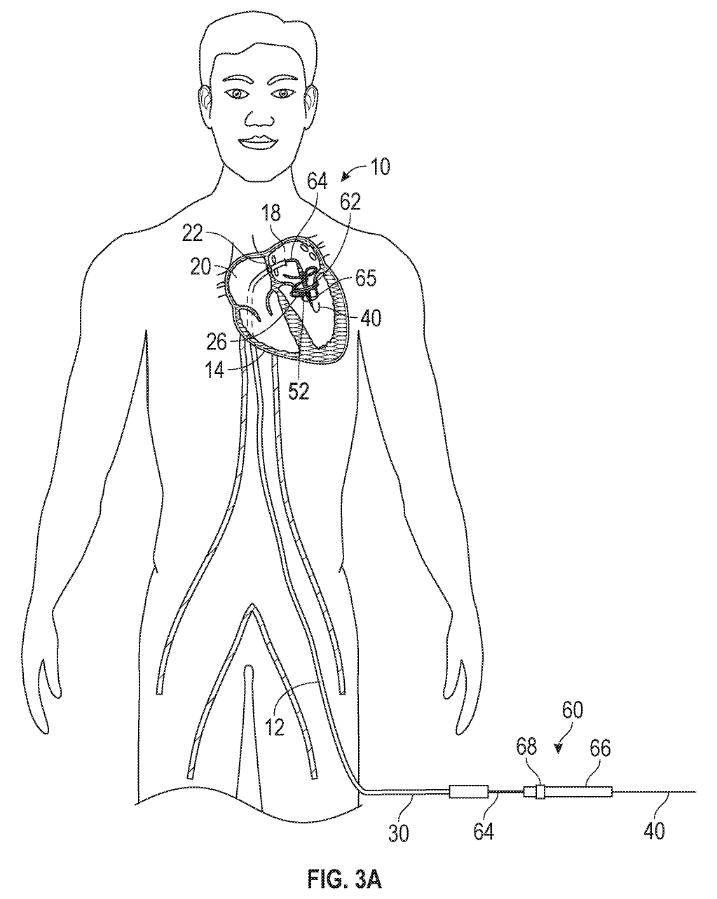

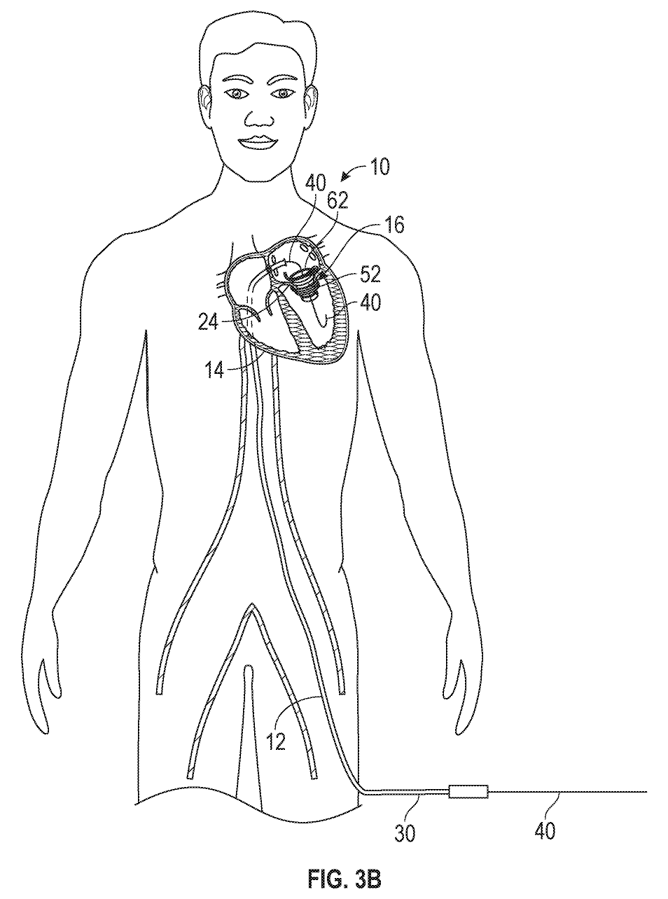

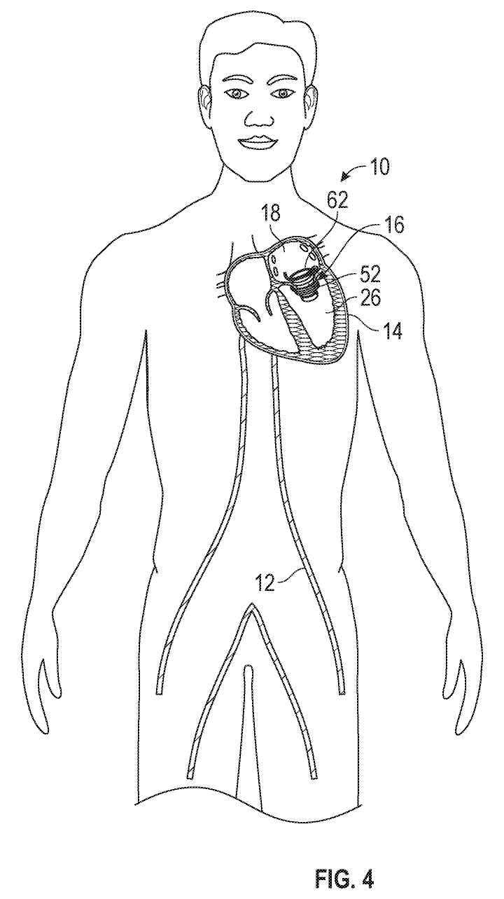

FIGS. 1-4 depict an example of a transcatheter heart valve replacement procedure (e.g., a mitral valve replacement procedure) which utilizes a docking device 52 and a prosthetic heart valve 62, according to one example. During the procedure, a user first creates a pathway to a patient’s native heart valve using a guide catheter 30 (FIG. 1). The user then delivers and implants the docking device 52 at the patient’s native heart valve using a docking device delivery apparatus 50 (FIG. 2A) and then removes the docking device delivery apparatus 50 from the patient 10 after implanting the docking device 52 (FIG. 2B). The user then implants the prosthetic heart valve 62 within the implanted docking device 52 using a prosthetic valve delivery apparatus 60 (FIG. 3A). Thereafter, the user removes the prosthetic valve delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient’s body to access the blood vessel 12. For example, in the example illustrated in FIG. 1, the user may make an incision in the patient’s groin to access a femoral vein. Thus, in such examples, the blood vessel 12 may be a femoral vein.

After making the incision at the blood vessel 12, the user may insert the guide catheter 30, the guidewire 40, and/or additional devices, such as an introducer device 100 (sometimes referred to as a dilator) through the incision and into the blood vessel 12. The guide catheter 30 (or “guide sheath”) is configured to facilitate the percutaneous introduction of various implant delivery devices (e.g., the docking device delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (e.g., the guide catheter 30, the docking device delivery apparatus 50, the prosthetic valve delivery apparatus 60, the introducer device 100, additional catheters, or the like) and their associated devices (e.g., docking device, prosthetic heart valve, and the like) to the implantation site within the heart 14, and thus may extend all the way through the blood vessel 12 and into a left atrium 18 of the heart 14 (FIG. 1) and, in some examples, through the native mitral valve 16 and into a left ventricle of the heart 14.

In some instances, a transseptal puncture device can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (e.g., through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10.

In some instances, the introducer device 100, can be inserted through a lumen of the guide catheter 30 prior to inserting the guide catheter 30 into the blood vessel 12. The introducer device 100 can include a tapered nosecone 102 that extends out a distal tip of the guide catheter 30 and that is configured to expand the initial incision in the atrial septum and to introduce the guide catheter 30 into the left atrium 18 over the guidewire 40, as shown in FIG. 1. Additionally, in some instances the introducer device 100 can include a handle 104 that extends out a proximal end of the guide catheter 30, and allows for the introducer device 100 to be removed from the guide catheter 30 after the initial incision has been expanded and the guide catheter 30 has been introduced into the left atrium 18. Once the guide catheter 30 reaches the left atrium 18, the user can remove the introducer device 100 and the guidewire 40, for example by pulling the introducer device 100 and/or the guidewire 40 in a proximal direction, from inside the guide catheter 30 and the patient 10. Thus, only the guide catheter 30 remains inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a docking device delivery apparatus 50 (which may also be referred to as an “implant catheter” and/or a “docking device delivery device”).

In general, the docking device delivery apparatus 50 comprises a delivery shaft 54, a handle 56, and a pusher assembly 58. The delivery shaft 54 is configured to be advanced through the patient’s vasculature (blood vessel 12) and to the implantation site (e.g., native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the docking device delivery apparatus 50 is configured to be gripped and/or otherwise held by the user, outside the body of the patient 10, to advance the delivery shaft 54 through the patient’s vasculature (e.g., blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (e.g., the native mitral valve 16). For example, the pusher assembly 58 is configured to be adjusted by the user to push the docking device 52 out of the distal end portion 53 of the delivery shaft 54. A shaft of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the docking device delivery apparatus 50 such that the docking device 52 can be released after being deployed at the native mitral valve 16.

Further details of the docking device delivery apparatus and its variants are described in International Publication No. WO2020/247907.

Referring again to FIG. 2A, after the guide catheter 30 is positioned within the left atrium 18, the user may insert the docking device delivery apparatus 50 (e.g., the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the blood vessel 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (e.g., pushing) the handle 56 of the docking device delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (e.g., the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (e.g., the portion of the docking device 52 shown in FIG. 2A that is configured to be positioned within a left ventricle 26 and/or on the ventricular side of the native mitral valve 16), the user may then deploy the remaining portion of the docking device 52 (e.g., an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the docking device delivery apparatus 50 from the docking device 52. Once the docking device 52 is disconnected from the docking device delivery apparatus 50, the user may retract the docking device delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient’s vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient’s vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (e.g., the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (e.g., pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (e.g., inflate the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 50, prosthetic valve delivery apparatus 60, guide catheter 30, and/or guidewire 40), docking devices (e.g., docking device 52), replacement heart valves (e.g., prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient’s vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

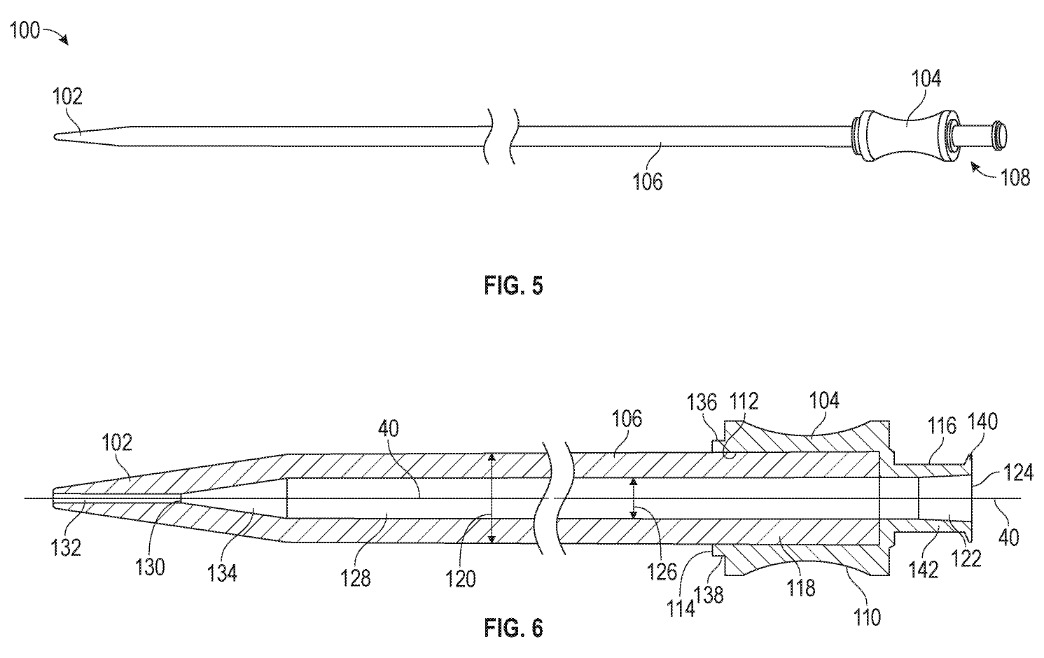

Turning now to FIG. 5 the introducer assembly 100 is shown in greater detail. The introducer assembly 100 comprises a tapered nosecone 102 and a handle 104 separated by a shaft 106. The introducer assembly 100 is used to expand or dilate the vasculature of a patient, such as an initial incision in the atrial septum, prior to the implantation of an implantable device. The introducer assembly 100 can also introduce other components of the delivery apparatus, such as the guide catheter 30 disclosed herein, across the atrial septum and into the left atrium as shown in FIG. 1. This ensures that the initial incision has sufficient diameter to accommodate the delivery apparatus and any subsequently implanted prosthetic device, such as a prosthetic heart valve and/or docking device.

As shown in FIGS. 5 and 6, the tapered nosecone 102 can have a conical geometry, in some instances. The tapered nosecone can comprise various other shapes having a relatively gradual or smooth transition in the outer diameter. The tapered nosecone 102 is located at the distal end of the introducer assembly 100 and can also include a bore 130 extending axially through the nosecone 102, as shown in FIG. 6. In some examples, the bore 130 can have a cylindrical first bore portion 132 positioned towards the distal end of the tapered nosecone 102, and a conical or tapered second bore portion 134 that extends between the first bore portion 132 and a channel 128 extending axially through the length of the shaft 106. Advantageously, this allows for the distally furthest portion of the bore 130 to have a smaller diameter to more closely conform to the diameter of the guidewire 40, while allowing the channel 128 extending axially through the shaft 106 to have a comparatively larger diameter.

In some instances, the dimensions of the introducer can be influenced by the need to sterilize and subsequently degas the introducer. For example, the introducer can be sterilized using ethylene oxide (EO). In such examples, it is desirable to have a sufficiently large inner lumen so that the sterilization fluid can flow through the inner lumen.

In some examples, the tapered nosecone 102 of the introducer assembly 100 can comprise a biocompatible polymeric material. Thus, the tapered end portion can penetrate the initial incision in the atrial septum 22 described herein. As the tapered nosecone 102 is advanced distally (that is, advanced through the initial incision), the increasing diameter will gradually expand the incision in the transatrial septum to a greater diameter such that the incision is capable of accommodating the guide sheath 30.

The handle 104 is positioned at the proximal end portion 108 of the introducer assembly 100 (that is, the end of the introducer assembly 100 closest to the user). As shown in FIG. 6, the handle 104 can comprise a main body 110 defining an internal chamber 112 that is open at both a distal end portion 114 and a proximal end portion 116 of the handle 104. As shown in FIG. 6, the chamber 112 can have a first inner diameter that can be sized to receive a proximal end portion 118 of the shaft 106. That is, the inner diameter of the chamber 112 can correspond an outer diameter 120 of the shaft 106, such that the proximal end portion 118 of the shaft 106 fits snugly into the chamber 112 when the handle 104 receives the shaft 106, as shown in FIG. 6. In lieu of or in addition to a frictional fit, the handle 104 can be coupled to the shaft 106 via adhesive, fasteners, overmolding, etc. In some instances, the handle 104 and the shaft 106 can be integrally formed (e.g., via molding) as a single, unitary component.

With continued reference to FIG. 6, the proximal end portion 116 of the handle 104 can comprise an internal bore 122 that extends between channel 128 extending through the shaft 106 and a mouth 124 located at the most proximal end of the handle 104. The bore 122 can, as shown in FIG. 6, have a diameter that corresponds to and is aligned with the diameter 126 of the channel 128 extending axially along the length of the shaft 106. In some examples, the bore 122 can taper radially outwardly from the distal end (i.e., the end abutting the channel 128) to the proximal end. Thus, the bore 122 can open the channel 128 of the shaft 106 to the outside environment, and allow for various components of the delivery apparatus, for example, the guidewire 40 described herein, to extend through the shaft 106 and the handle 104 and out of the introducer assembly 100. Advantageously, this allows the guidewire 40 to be withdrawn through the introducer assembly 100 as described herein.

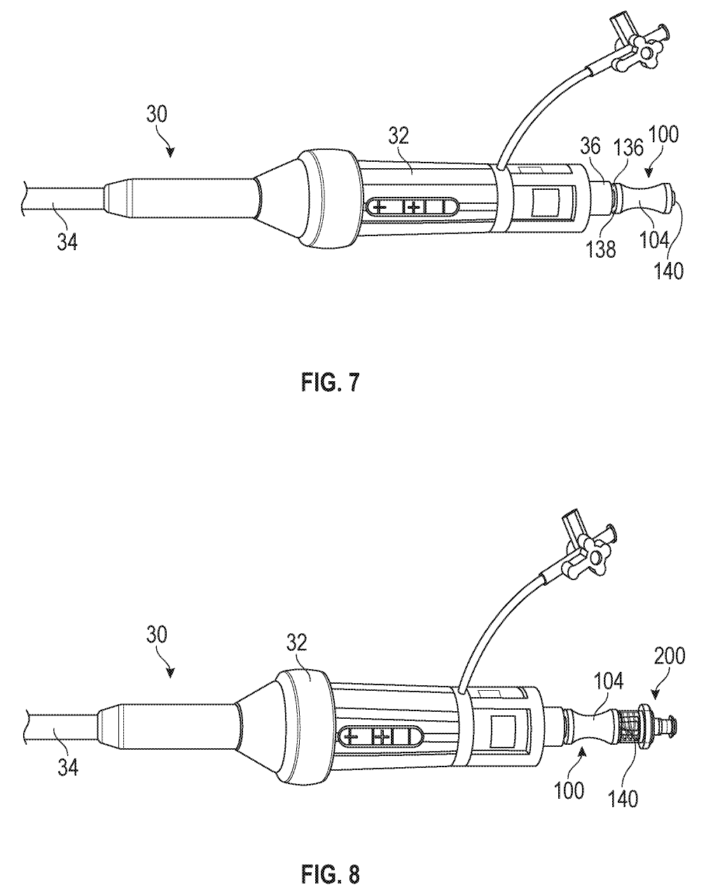

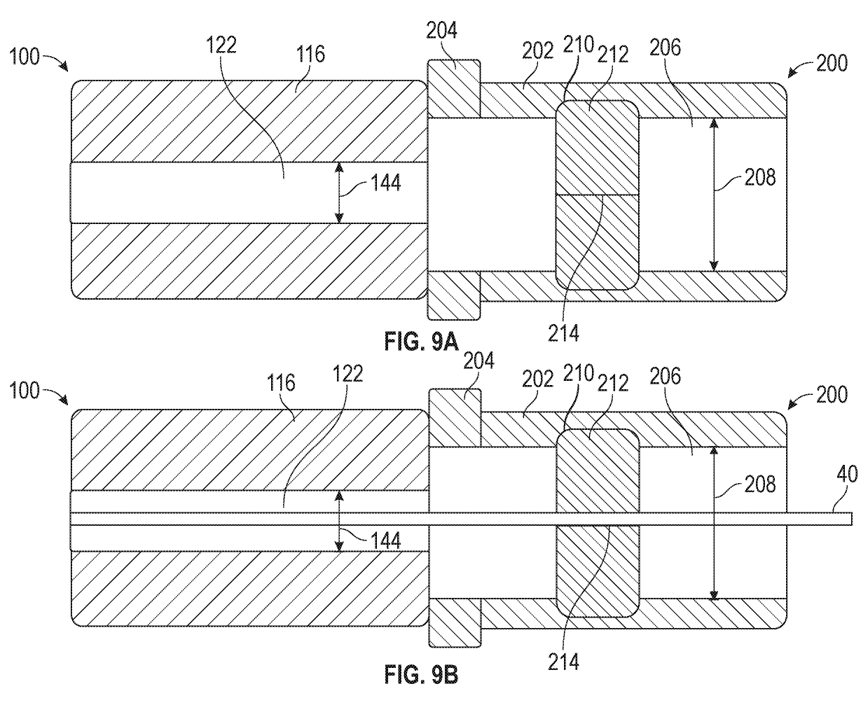

As shown in FIG. 6, the distal end portion 114 of the handle 104 can have a circumferential ridge 136 extending around the external diameter of the shaft 106. This forms an annular step 138 at the distal end portion 114 of the handle 104, which, as discussed in greater detail herein and shown in FIGS. 7 and 8, can align the introducer assembly 100 with a portion of the guide sheath 30, such as a port in the handle 32 of the guide sheath 30 disclosed in further detail herein. Advantageously, this can ensure correct orientation and positioning of the introducer assembly 100 relative to the guide sheath 30, while preventing any inadvertent over-insertion of the introducer assembly 100 into the guide sheath 30.

The proximal end portion 116 of the handle 104 can also include an annular flange 140 located around the mouth 124 of the bore 122. The flange 140 can extend radially outwards from a cylindrical body 142 of the proximal end portion 116 of the handle 104. As discussed in greater detail herein, and shown in FIG. 8, the flange 140 can be configured to permit the attachment of a seal assembly, such as the seal assembly 200 discussed in greater detail herein.

As shown in FIGS. 5 and 6, the handle 104 can have a curved or hourglass profile. Advantageously, the curved or hourglass profile can facilitate gripping the handle 104, such as, for example, during the removal of the introducer assembly from a guide sheath. It is to be understood, however, that in some examples the overall shape of the handle 104 can omit the curved or hourglass profile, having substantially parallel sides. In some examples, the handle 104 can also have other geometries which facilitate gripping and manipulation of the handle 104 by a medical professional during and implantation process.

While FIG. 2 shows that the handle 104 and the shaft 106 can be formed as separate components and subsequently fit together to form the introducer assembly 100, it is to be appreciated that, in some examples, the introducer assembly can be formed as a unitary body. That is, the handle 104, the shaft 106, and the tapered nosecone 102 can be formed as a single piece.

Turning now to FIGS. 7 and 8, the introducer assemblies disclosed herein, such as the introducer assembly 100, are configured for use with a guide catheter, such as the guide catheter 30 disclosed herein. When used with a guide catheter, such as the guide catheter 30 illustrated, the nosecone 102 and the shaft 106 of the introducer assembly 100 can be inserted into a bore in the handle 32 of the guide catheter 30 and extended down the shaft 34 of the guide catheter 30. Thus, nosecone 102 of the introducer assembly 100 can advance through the vasculature of the patient along with a distal end portion of the guide catheter 30, towards the desired implantation site, as shown in FIG. 1.

In some examples, such as that shown in FIGS. 7 and 8, the handle 104 of the introducer assembly 100 can be configured to engage with a dock 36 located at the proximal end of the guide catheter 30. As shown, the circumferential ridge 136 can be sized to fit inside the internal bore of the dock 36, and the annular step 138 can come into contact with the proximal end of the guide catheter 30. This limits the progress of the introducer assembly 100 in the distal direction and can prevent over-insertion of the introducer assembly 100.

The geometry of introducer assembly 100 and particularly the length of the shaft 106 can, in certain examples, be selected to correspond to the length of the shaft 34 of the guide catheter 30. Thus, as shown in FIG. 1, the nosecone 102 of the introducer assembly 100 can protrude from a distal end portion of the shaft 34 of the guide catheter 30 when the introducer assembly 100 is fully inserted into the guide catheter 30 (that is, when the circumferential ridge 136 and annular step 138 are engaged with the proximal end of the guide catheter 30).

After the shaft 34 of the guide catheter 30 has been advanced to the desired implantation location (for example, into the left atrium 18 as shown in FIG. 1), the introducer assembly 100 can be removed from the patient by withdrawing the handle 104 (and thus the introducer assembly 100) in the proximal direction. This withdraws the shaft 106 and the nosecone 102 of the introducer assembly 100 through the shaft 34 of the guide catheter 30.

As the introducer assembly 100 is withdrawn through the shaft 34 of the guide catheter 30, the space formerly filled by the introducer assembly 100 will gradually fill with blood. However, the orientation of the guide catheter can, in some examples, result in the shaft 34 of the guide catheter 30 to fill slowly. This backflow of blood is desirable, because, when the shaft 34 of the guide catheter 30 is adequately backfilled, the patient’s blood functionally seals off the vasculature against the introduction of air into the vasculature of the patient. However, because blood may be slow to backfill the shaft 34 of the guide catheter 30, the removal of the introducer assembly 100 is preferably accomplished without inadvertent introduction of air.

In some examples, it may be necessary to remove the guidewire 40 along with or prior to the removal of the introducer assembly 100. In such examples, the guidewire 40 can be moved in the proximal direction in advance of or along with the removal of the introducer assembly 100. However, because it is desirable to prevent air from entering the introducer assembly 100, such as through the bore 122 of the handle 104 and into the channel 128 of the shaft 106, it may also be desirable to include a seal assembly to prevent the introduction of air into the introducer assembly 100. Example seal assemblies include a Touhy seal or a stopcock, as previously described herein. In some examples, to simplify the removal of the guidewire 40, a passive seal assembly 200, can be attached to the proximal end portion of the introducer assembly 100 as shown in FIG. 8. For example, the seal assembly 200 can be attached to the flange 140, closing off the bore 122 of the handle 104 from the outside environment.

The seal assembly 200, as shown in more detail in FIGS. 9A and 9B, comprises a cylindrical housing 202. The cylindrical housing 202 can further include a flange 204, which can be configured to attach the seal assembly 200 to the introducer assembly 100, such as at the flange 140 previously described and shown in FIG. 6.

The seal assembly 200 can also include a bore 206, extending axially through the length of the seal assembly 200. As shown in FIGS. 9A and 9B, the bore 206 can have a diameter 208 that is selected to be larger than a diameter 144 of the bore 122 extending axially through the distal end portion 116 of the introducer assembly 100.

The cylindrical housing 202 of the seal assembly 200 can further comprise an annular internal groove 210 which is configured to receive a passive sealing element 212. The passive sealing element 212 is, in some examples, made of a resilient material, such as silicone rubber, polyurethane, or a combination thereof, and can have an uncompressed diameter that is larger than the diameter of the annular internal groove 210, such that the sealing element 212 will be at least partially radially compressed by radial stresses imposed by the constraint of the annular internal groove 210.

The passive sealing element 212 can comprise an axially extending slit 214 positioned substantially in the center of the sealing element 212. The axially extending slit 214 can be movable between a closed configuration (shown in FIG. 9A) and an open configuration (shown in FIG. 9B). In some examples, this is done by introducing a wire, such as the guidewire 40 described herein, to the slit 214 or removing the wire from the slit 214, as shown in FIG. 9B.

More specifically, because the sealing element 212 is retained under compressive radial stress, the slit 214 is held in a closed configuration (that is, the internal walls of the slit can be pressed against one another by the compressive radial stresses acting on the sealing element) when there is no object extending through the sealing element 212. However, because the sealing element 212 comprises a resilient material, such as those listed herein, the introduction of a wire, such as the guidewire 40, will introduce a radially expansive stress to the slit 214, and will stretch the slit 214 into an open configuration to accommodate the guidewire 40. Because the compressive stresses imparted by the annular internal groove 210 are not relieved when the wire is passed through the slit 214 and across the sealing element 212, the internal walls of the slit will still be pressed inwards against the outer surface of the guidewire 40. In this way, an airtight seal may be maintained between the guidewire 40 and the sealing element 212 while the guidewire 40 extends across the sealing element 212.

When the guidewire 40 is removed from the sealing element 212, such as when the guidewire 40 is withdrawn to facilitate the withdrawal of the introducer assembly 100 from the guide catheter 30, the radially expansive forces imparted by the guidewire 40 on the interior of the slit 214 are relieved. This causes the slit 214 to return to the closed configuration behind the guidewire 40 as it is removed, and an airtight seal is restored across the slit 214 as it returns to the closed configuration. In this way, the guidewire 40 can be withdrawn from the slit 214 without disrupting the airtight seal provided by the sealing element 212. This allows a medical professional to simply remove the introducer assembly 100 together with the guide wire 40 from the guide catheter 30 or remove the guidewire 40 and the introducer assembly 100 from the guide catheter 30 sequentially without needing to manipulate a sealing apparatus such as a stopcock or a Touhy seal, as described herein. In this way, the passive sealing assembly disclosed herein reduce the number of steps the user needs to perform and/or reduces the likelihood of an air embolism.

In some examples, the guidewire 40 is inserted into the introducer assembly 100 through the distal nosecone 102 (or more specifically, through the first bore portion 132 extending through the tip of the tapered nose cone 102). The guidewire 40 can then be advanced through the introducer assembly 100 in the proximal direction, and out of the introducer assembly 100 through the bore 122 in the handle 104. In such examples, it may be challenging to properly align the guidewire 40 with the slit 214 in the sealing element 212 because the guidewire 40 is being inserted into the nosecone 102 of the introducer assembly 100 and feed proximally through the sealing element 212 (i.e., left to right in the orientation depicted in FIGS. 9A-9B).

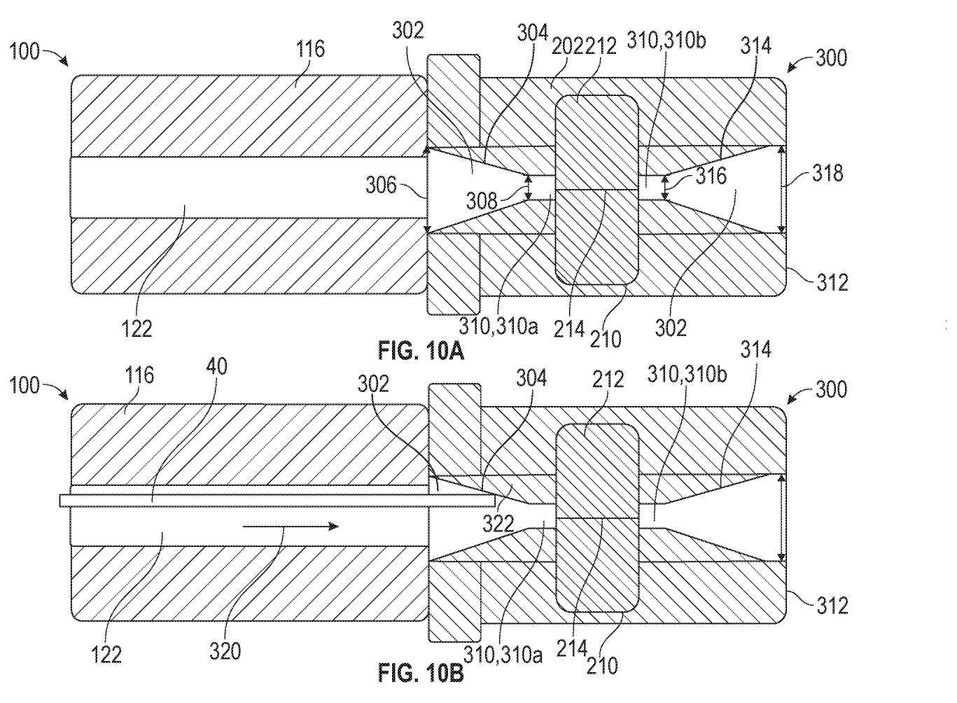

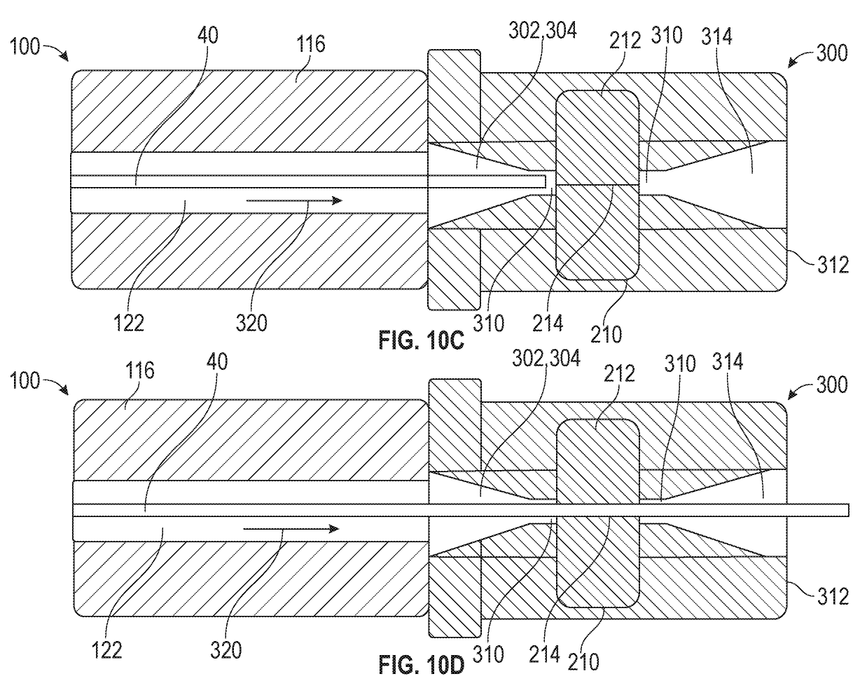

To improve the alignment of the guidewire 40 and the slit 214 in the sealing element 212, a seal assembly having one or more internally positioned guide features can be used. An example seal assembly 300 with one or more internally positioned guide features is illustrated in FIGS. 10A-10D. The seal assembly 300 can have the same or substantially the same basic structure and configuration as the seal assembly 200 disclosed herein and described in relation to FIGS. 9A and 9B and can function in the same way as disclosed for the seal assembly 200, except for the differences described below.

The seal assembly 300 can include an axially extending internal bore 302 that connects the bore 122 of the distal portion 114 of the introducer assembly 100 with the outside environment when the seal assembly 300 is attached to the introducer assembly 100. As shown in FIG. 10A, the internal bore 302 comprises a first tapered section 304 that narrows from a larger first diameter 306 at the distal end of the seal assembly 300 to a smaller second diameter 308 as the tapered section 304 extends towards the sealing element 212. In some examples, the first diameter 306 can be at least substantially equal or greater than the diameter of the bore 122 extending through the proximal portion 116 of the handle 104 of the introducer assembly 100. This helps to ensure that there is no burr or seam between the bore 122 and the first tapered section 304 of the bore 302, which can catch or snag an advancing wire, such as the guidewire 40, as it is advanced in the proximal direction into the seal assembly 300.

The internal bore 302 also comprises a narrow central channel 310, which in some examples, can have the same second diameter 308 as the proximal end portion of the tapered bore 304. The central channel 310 extends axially from the tapered bore 304 towards a proximal end 312 of the seal assembly 300, and in some examples can be partitioned into a first channel segment 310a and a second channel segment 310b by the sealing element 212.

In some examples, the internal bore 302 can further comprise an optional second tapered section 314, which extends axially from the central channel 310 (particularly, from the second channel segment 310b as shown in FIGS. 10A-10D), to the proximal end 312 of the seal assembly 300. The second tapered section 314 can, as shown in FIG. 10A, widen from a smaller third diameter 316 adjacent to the central channel 310 to a larger fourth diameter 318 adjacent to the proximal end 312 of the seal assembly 300. In some examples, the third diameter 316 can be the same as the second diameter 308, and the fourth diameter 318 can be the same as the first diameter 306. Advantageously, the inclusion of the second tapered section 314 can facilitate an alternative guidewire introduction procedure in which the guidewire 40 is inserted from the proximal end of the delivery assembly (and thus benefits from a tapered bore guide located further in the proximal direction than the sealing element 212).

As illustrated in FIGS. 10B-10C, the tapered section 304 of the bore 302 can facilitate the introduction of the guidewire 40 to the seal assembly 300 and can ensure that the guidewire 40 is properly aligned with the slit 214 in the sealing element 212.

Particularly, as shown in FIG. 10B, as the guidewire 40 is advanced in the proximal direction, indicated by the arrow 320 (that is, from the distal end of the introducer assembly 100, towards the proximal end 114 of the introducer assembly 100, and into the seal assembly 300), the guidewire 40 may, in some instances, drift towards a radial extreme of the bore 122. When the guidewire 40 passes from the bore 122 and into the tapered section 304 of the bore 302 while positioned towards a radial extreme of the bore 122, it can impinge upon a sloped internal wall 322 of the tapered section 304.

As shown in FIG. 10C, as the guidewire 40 is further advanced in the proximal direction, indicated by the arrow 320, the sloped internal wall 322 of the tapered section 304 pushes the guidewire 40 further towards the radial center of the bore 302, such that it can pass into the central channel 310 of the bore 302. This aligns the proximal end of the guidewire 40 with the slit 214 in the sealing element 212, such that as the guidewire 40 is further advanced in the proximal direction, as shown in FIG. 10D, the guidewire 40 will pass through the slit 214, which expands from the closed configuration to the open configuration in a fashion substantially identical to that described herein for the seal assembly 200 in order to accommodate the guidewire 40.

Thereafter, the guidewire 40 can be advanced in the proximal direction such that the distal end portion of the guidewire 40 extends out of the proximal end 312 of the seal assembly 300. This allows a physician to withdraw the guidewire 40 from the seal assembly 300 (and, therefore, also from the introducer assembly 100) in the proximal direction by pulling on the proximal end of the guidewire 40. This causes the slit 214 in the sealing element 212 to return to the closed configuration from the open configuration as the guidewire 40 is fully removed from the seal assembly 300 and the introducer assembly 100. As described herein in relation to the seal assembly 200, this allows for the guidewire 40 to be withdrawn from the introducer assembly 100 without breaking the seal provided by the sealing element 212, thus preventing the introduction of air into any part of the bore extending through the center of the introducer assembly 100. It also allows the introducer assembly 100 and the guide wire 40 to be withdrawn together without introducing air into the system.

In this way, the sealing assemblies described herein, such as sealing assemblies 200 and 300 improve the functionality of the introducer assemblies, such as the introducer assembly 100, described herein. By including a seal that moves passively between the open and closed configuration, without either disrupting the airtight seal, or requiring additional input steps from an operating medical professional, the seal assemblies 200, 300 improve the security of the introducer assembly 100 against inadvertent air introduction. Moreover, these seal assemblies passively form an airtight seal with a guidewire, such as guidewire 40, when it lies across the seal, such as the sealing element 212, also without the need of any additional input from the operating medical professional.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An introducer assembly comprising a dilator comprising a nosecone, a handle, a tubular shaft extending between the nosecone and a first axial end portion of the handle, and an internal channel extending axially through the nosecone, shaft, and handle; and a seal assembly attached to a second axial end portion of the handle, the seal assembly comprising a housing, a bore extending axially through the housing, and a sealing element disposed in the bore; wherein the dilator and the seal assembly are configured to receive a guidewire extending axially through the dilator and the seal assembly, and wherein the sealing element comprises a self-sealing channel configured to receive a guidewire, movable from a closed state to an open state when the guidewire is inserted into the self-sealing channel, and movable from the open state to the closed state when the guidewire is removed from the internal channel.

Example 2. The introducer assembly of any example herein, particularly example 1, wherein the bore of the seal assembly further comprises a first bore section having a first diameter, a second bore section having a second diameter that is smaller than the first diameter, and a tapered section extending between the first bore section and the second bore section.

Example 3. The introducer assembly of any example herein, particularly example 2, wherein the sealing element is positioned across the second bore section such that the self-sealing channel is positioned within the second bore section.

Example 4. The introducer assembly of any example herein, particularly examples 2-3, wherein the tapered section is a first tapered bore section, the bore further comprises a third bore section having a third diameter greater than the second diameter, and the bore further comprises a second tapered section extending between the second bore section and the third bore section.

Example 5. The introducer assembly of any example herein, particularly examples 1-3, wherein the housing of the seal assembly further comprises an annular groove and wherein the sealing element is positioned within the annular groove.

Example 6. The introducer assembly 5, wherein the sealing element in an uncompressed state has an outer diameter that is greater than an inner diameter of the annular groove.

Example 7. The introducer assembly of any example herein, particularly examples 1-6, wherein the sealing element is retained in a state of radial compression within the housing of the seal assembly.

Example 8. The introducer assembly of any example herein, particularly examples 1-7, wherein the seal assembly provides an airtight seal across the self-sealing channel in both the open state and the closed state.

Example 9. The introducer assembly of any example herein, particularly examples 1-8, wherein the self-sealing channel recovers resiliently from the open state to the closed state when the guidewire is removed from the self-sealing channel.

Example 10. The introducer assembly of any example herein, particularly examples 1-9, wherein the sealing element comprises silicone, polyurethane, or a combination thereof.

Example 11. The introducer assembly of any example herein, particularly examples 1-10, wherein the first diameter of the first bore section is larger than a channel diameter of the internal channel of the dilator.

Example 12. A seal assembly for an introducer, comprising a seal housing having an annular body comprising an internal circumferential groove and one or more radially tapered internal wall portions; an internal bore extending axially from a first end portion of the seal housing to a second end portion of the seal housing; and a resilient sealing element disposed in the internal groove and comprising an axially extending slit circumferentially aligned with the internal bore; wherein the axially extending slit is movable between an open state when a guidewire is inserted into the slit and a closed state when the guidewire is withdrawn from the slit, and wherein the seal assembly is configured to attach to an end portion of an introducer assembly comprising a hollow shaft with an internal channel.

Example 13. The seal assembly of any example herein, particularly example 12, wherein when the slit is in the open state, it forms an airtight seal with the guidewire.

Example 14. The seal assembly of any example herein, particularly examples 12-13, wherein when the slit is in the closed state, the internal wall of the slit forms an airtight seal with itself.

Example 15. The seal assembly of any example herein, particularly examples 12-14, wherein the sealing element is retained in a partially radially compressed state by the housing of the seal assembly.

Example 16. The seal assembly of any example herein, particularly examples 12-15, wherein the one or more radially tapered internal wall portions define a first conical bore section, a second conical bore section, and a cylindrical bore section extending between the first conical bore section and the second conical bore section.

Example 17. The seal assembly of any example herein, particularly example 16, wherein the sealing element extends across the cylindrical bore section.

Example 18. A delivery system comprising a guide catheter comprising a tubular shaft having an internal lumen; a removable guidewire configured to extend through the internal lumen of the guide catheter; an introducer comprising a nosecone, a shaft, a handle, and an internal bore extending axially through the nosecone, the shaft, and the handle; and a seal assembly attached to the handle of the introducer, comprising a seal housing, a seal element disposed within the seal housing, and one or more beveled guides defining a guide channel that extends axially through the housing of the seal assembly and narrows from a first diameter at a first channel end to a second diameter smaller than the first diameter at a second channel end; wherein the internal bore of the introducer and the guide channel of the seal assembly are configured to receive the guidewire, wherein the seal assembly comprises an axially extending self-sealing channel that is configured to receive the guidewire, and wherein the self-sealing channel is movable from a closed state to an open state by introducing a wire into the self-sealing channel.

Example 19. The delivery system of any example herein, particularly example 18, wherein the seal assembly wherein when the self-sealing channel is in the open state, an internal diameter of the self-sealing channel forms an airtight seal with the wire.

Example 20. The delivery system of any example herein, particularly examples 18-19, wherein the guide channel is a first guide channel and the seal housing further comprises as second guide channel that narrows from a third diameter at a third channel end to a fourth diameter smaller than the third diameter at a fourth channel end, and wherein the seal assembly is positioned between the second channel end and the fourth channel end.

Example 21. An introducer assembly, a seal assembly, or a delivery system of any preceding example, wherein the introducer assembly, the seal assembly, or the delivery system is sterilized.

Example 22. A method comprising sterilizing the introducer assembly, the seal assembly, or the delivery system of any preceding example.

The features described herein with regard to any example can be combined with other features described in any one or more of the other examples, unless otherwise stated. For example, any one or more of the features of one introducer assembly can be combined with any one or more features of another introducer assembly. As another example, any one or more features of one delivery system or seal assembly can be combined with any one or more features of another delivery system or seal assembly.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. An introducer assembly comprising:
a dilator comprising a nosecone, a handle, a tubular shaft extending between the nosecone and a first axial end portion of the handle, and an internal channel extending axially through the nosecone, shaft, and handle; and
a seal assembly attached to a second axial end portion of the handle, the seal assembly comprising a housing, a bore extending axially through the housing, and a sealing element disposed in the bore;
wherein the dilator and the seal assembly are configured to receive a guidewire extending axially through the dilator and the seal assembly, and
wherein the sealing element comprises a self-sealing channel configured to receive a guidewire, movable from a closed state to an open state when the guidewire is inserted into the self-sealing channel, and movable from the open state to the closed state when the guidewire is removed from the internal channel.

2. The introducer assembly of claim 1, wherein the bore of the seal assembly further comprises a first bore section having a first diameter, a second bore section having a second diameter that is smaller than the first diameter, and a tapered section extending between the first bore section and the second bore section.

3. The introducer assembly of claim 2, wherein the sealing element is positioned across the second bore section such that the self-sealing channel is positioned within the second bore section.

4. The introducer assembly of claim 2, wherein the tapered section is a first tapered bore section, the bore further comprises a third bore section having a third diameter greater than the second diameter, and the bore further comprises a second tapered section extending between the second bore section and the third bore section.

5. The introducer assembly of claim 1, wherein the housing of the seal assembly further comprises an annular groove and wherein the sealing element is positioned within the annular groove.

6. The introducer assembly of claim 5, wherein the sealing element in an uncompressed state has an outer diameter that is greater than an inner diameter of the annular groove.

7. The introducer assembly of claim 1, wherein the sealing element is retained in a state of radial compression within the housing of the seal assembly.

8. The introducer assembly of claim 1, wherein the seal assembly provides an airtight seal across the self-sealing channel in both the open state and the closed state.

9. The introducer assembly of claim 1, wherein the self-sealing channel recovers resiliently from the open state to the closed state when the guidewire is removed from the self-sealing channel.

10. The introducer assembly of claim 1, wherein the sealing element comprises silicone, polyurethane, or a combination thereof.

11. The introducer assembly of claim 1, wherein the first diameter of the first bore section is larger than a channel diameter of the internal channel of the dilator.

12. A seal assembly for an introducer, comprising:
a seal housing having an annular body comprising an internal circumferential groove and one or more radially tapered internal wall portions;
an internal bore extending axially from a first end portion of the seal housing to a second end portion of the seal housing; and
a resilient sealing element disposed in the internal groove and comprising an axially extending slit circumferentially aligned with the internal bore;
wherein the axially extending slit is movable between an open state when a guidewire is inserted into the slit and a closed state when the guidewire is withdrawn from the slit, and
wherein the seal assembly is configured to attach to an end portion of an introducer assembly comprising a hollow shaft with an internal channel.

13. The seal assembly of claim 12, wherein when the slit is in the open state, it forms an airtight seal with the guidewire.

14. The seal assembly of claim 12, wherein when the slit is in the closed state, the internal wall of the slit forms an airtight seal with itself.

15. The seal assembly of claim 12, wherein the sealing element is retained in a partially radially compressed state by the housing of the seal assembly.

16. The seal assembly of claim 12, wherein the one or more radially tapered internal wall portions define a first conical bore section, a second conical bore section, and a cylindrical bore section extending between the first conical bore section and the second conical bore section.

17. The seal assembly of claim 16, wherein the sealing element extends across the cylindrical bore section.

18. A delivery system comprising:
a guide catheter comprising a tubular shaft having an internal lumen;
a removable guidewire configured to extend through the internal lumen of the guide catheter;
an introducer comprising a nosecone, a shaft, a handle, and an internal bore extending axially through the nosecone, the shaft, and the handle; and
a seal assembly attached to the handle of the introducer, comprising a seal housing, a seal element disposed within the seal housing, and one or more beveled guides defining a guide channel that extends axially through the housing of the seal assembly and narrows from a first diameter at a first channel end to a second diameter smaller than the first diameter at a second channel end;
wherein the internal bore of the introducer and the guide channel of the seal assembly are configured to receive the guidewire,
wherein the seal assembly comprises an axially extending self-sealing channel that is configured to receive the guidewire, and
wherein the self-sealing channel is movable from a closed state to an open state by introducing a wire into the self-sealing channel.

19. The delivery system of claim 18, wherein the seal assembly wherein when the self-sealing channel is in the open state, an internal diameter of the self-sealing channel forms an airtight seal with the wire.

20. The delivery system of claim 18, wherein the guide channel is a first guide channel and the seal housing further comprises as second guide channel that narrows from a third diameter at a third channel end to a fourth diameter smaller than the third diameter at a fourth channel end, and wherein the seal assembly is positioned between the second channel end and the fourth channel end.
